(19) United States
(12) Patent Application Publication
(10) Pub. No.: US 2018/0037976 A1
(43) Pub. Date: Feb. 8, 2018

(54) Preparation method and application of three-dimensional interconnected porous magnesium-based material

(71) Applicant: SHANGHAI JIAO TONG UNIVERSITY, Shanghai (JP)

(72) Inventors: Guangyin Yuan, Shanghai (CN); Gaozhi Jia, Shanghai (CN)

(21) Appl. No.: 15/552,260

(22) PCT Filed: Jan. 25, 2016
(86) PCT No.: PCT/CN2016/071982

§ 371 Date: Aug. 18, 2017

(30) Foreign Application Priority Data
Feb. 25, 2015 (CN) 201510087314.4
Jul. 7, 2015 (CN) 201510395799.3

(51) Int. Cl. C22C 1/08; A61L 27/04; A61L 27/56; C22C 23/04

(52) U.S. Cl. CPC C22C 1/08; C22C 23/04; A61L 27/047; A61L 27/56

(57) ABSTRACT

A preparation method and an application of a three-dimensional interconnected porous magnesium-based material are provided. The method includes steps of: preparing a porous titanium preform or a porous iron preform; introducing a molten magnesium-based metal into the porous titanium preform or the porous iron preform through pressure infiltration, and obtaining a porous magnesium-based material precursor; and washing the porous magnesium-based material precursor, and obtaining the porous magnesium-based material.

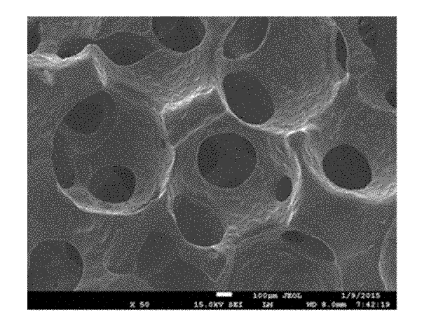

### CROSS REFERENCE OF RELATED APPLICATION

This is a U.S. National Stage under 35 U.S.C 371 of the International Application PCT/CN2016/071982, filed Jan. 25, 2016, which claims priority under 35 U.S.C. 119(a-d) to CN 201510087314.4, filed Feb. 25, 2015, and CN 201510395799.3, filed Jul. 7, 2015.

### BACKGROUND OF THE PRESENT INVENTION

### Field of Invention

The present invention belongs to a technical field of material preparation, relating to a design method of a three-dimensional open-cell porous material and more particularly to a preparation method and an application of a three-dimensional interconnected porous magnesium-based material.

### Description of Related Arts

In the biomedical metallic implant materials, because of the characteristics of good mechanical properties, biocompatibility and in vivo degradability, magnesium and its alloys have gained the wide attention and been widely studied in worldwide, and become the ideal materials of new-generation endosseous implants, intravascular stents, and dental and plastic implants, honored as the revolutionary metallic biomaterials. The porous magnesium-based biomaterial having the three-dimensional interconnected network structure not only serves as the tissue filler at the implant position, but also facilitates the ingrowth of the vessel and the surrounding tissue because of the own pore structure, so that the implant is avoided to be loose and fall off. Moreover, the porous magnesium-based biomaterial has a feature of body fluid transportation that during the reparative or plastic process of the implant position, the porous magnesium-based biomaterial is gradually degraded and absorbed, which achieves the autologous repairing effect. Furthermore, through controlling the pore characteristics of the porous material, the mechanical strength and the elasticity modulus of the implant are adjusted, so as to match with the performance of the autologous tissue.

At present, most of the researchers adopt the powder sintering method to prepare the porous magnesium and magnesium alloy. In order to increase the porosity and the interconnectivity, the pore-forming agents, such as NH₄HCO₃, CO(NH₂)₂, NaCl and methyl cellulose are usually added into the metallic powders. During the powder sintering process, because the morphology of the particles of the pore-forming agents is not uniform, it is failed to establish effective fusion points between the particles. As a result, the powder sintering method fails to guarantee the uniformity of the pore morphology and the interconnectivity of the pore structure. Moreover, during the removal process of the pore-forming agents, the residual of the pore-forming agents cannot be completely removed, and the pore-forming agents will corrode the magnesium matrix metal. Therefore, a new preparation method of the porous magnesium and magnesium alloy is necessary, which completely solves the problems in the conventional preparation of the porous magnesium and magnesium alloy, achieves the uniform pore distribution and the controllable mechanical properties, pore morphology and pore size, has the good interconnectivity and especially has no negative effect on the porous magnesium matrix during the preparation process.

### SUMMARY OF THE PRESENT INVENTION

The present invention provides a preparation method and an application of a three-dimensional interconnected porous magnesium-based material, so as to overcome above defects in prior arts. The obtained porous magnesium-based material is degradable open-cell porous magnesium or a degradable open-cell porous magnesium alloy.

The present invention firstly provides a preparation method of a three-dimensional interconnected porous magnesium-based material, comprising steps of:

preparing a porous titanium preform or a porous iron preform;

introducing a molten magnesium-based metal into the porous titanium preform or the porous iron preform through pressure infiltration, and obtaining a porous magnesium-based material precursor; and

washing the porous magnesium-based material precursor, and obtaining the porous magnesium-based material.

Preferably, the porous titanium preform or the porous ion preform is prepared through any preparation technology can bind metallic particles, such as cold press forming, hot isostatic pressing sintering, microwave sintering and spark plasma sintering.

Further preferably, preparing the porous titanium preform or the porous iron preform through the spark plasma sintering comprises steps of: under a pressure of 5-50 MPa, heating titanium particles or iron particles to a temperature of 600-1000° C. with a temperature increase rate of 10-100° C./min; keeping the pressure and the temperature, and then sintering; and, obtaining the porous titanium preform or the porous iron preform.

Further preferably, a particle size range of the titanium particles or the iron particles is in a range of 10-10000 μm. The titanium particles or the iron particles have a single particle size or various particle sizes for a mixed use.

Preferably, introducing the molten magnesium-based metal into the porous titanium preform or the porous iron preform through the pressure infiltration comprises steps of: under a pressure of 0.1-10 MPa, at a temperature of 650-750° C., pouring the molten magnesium-based metal into the porous titanium preform or the porous iron preform, and filling gaps of the porous titanium preform or the porous iron preform with the molten magnesium-based metal.

Preferably, washing the porous magnesium-based material precursor comprises steps of: acid washing through immersing the porous magnesium-based material precursor into a hydrofluoric acid solution, thereafter processing the porous magnesium-based material precursor with ultrasonic washing through an ultrasonic washing buffer solution, and repeating acid washing and ultrasonic washing for at least 3 times.

Preferably, the magnesium-based metal comprises following components by weight percentage of: magnesium: 70-100 wt. %; zinc: 0-30 wt. %; neodymium: 0-5 wt. %; yttrium: 0-10 wt. %; gadolinium: 0-10 wt. %; zirconium: 0-1 wt. %; calcium: 0-2 wt. %; aluminum: 0-9 wt. %; manganese: 0-1 wt. %; and arsenic: 0-2 wt. %.

The present invention further provides a porous magnesium-based material prepared by the above method, wherein: the porous magnesium-based material has a plurality of cavities therein, and the cavities are intercommunicated with each other through interconnected pores.

Preferably, the porous magnesium-based material has a porosity of 60-95%, a compressive strength of 1-30 MPa and an elasticity modulus of 0.05-1.5 GPa; and the interconnected pores have a pore size in a range of 2-5000 μm.

The present invention further provides an application of the above porous magnesium-based material in bone tissue engineering scaffolds and other engineering components of a magnesium alloy porous structure requiring characteristics of sound attenuation, sound absorption, noise reduction, shock absorption, thermal insulation, filtration and anti-collision.

Compared with prior arts, the present invention has following beneficial effects.

Firstly, the preparation method provided by the present invention has a simple process, an easy operation and no pollution. The open-cell porous structure prepared by the preparation method has the uniformly-distributed interconnected pores, the controllable pore morphology and pore size, the high porosity, and no closed pores and pore-forming agent residual.

Secondly, through selecting the titanium or iron particles of difference sizes (with a particle shape being spherical, elliptic, cubic or any other shape), adopting any preparation technology can bind the metallic particles, such as spark plasma sintering, microwave sintering, hot isostatic pressing sintering and cold press forming, and controlling a binding process between the metallic particles by adjusting process parameters such as the sintering temperature, pressure and time, the open-cell porous titanium or iron preform with the controllable pore size and interconnectivity is realized. Moreover, through the pressure infiltration, the control of the pore characteristics of the open-cell porous magnesium and magnesium alloy is indirectly realized.

Thirdly, the present invention adopts the hydrofluoric acid solution as a corrosion remover. The hydrofluoric acid reacts chemically with magnesium, and then forms a layer of compact magnesium fluoride film on the surface of the magnesium matrix. The film avoids the further corrosion of the hydrofluoric acid to magnesium, and meanwhile a chemical corrosion reaction occurs between the film and the titanium or iron preform, so as to rapidly remove the preform and meanwhile well protect the integrity and purity of the matrix structure of the open-cell porous magnesium and magnesium alloy.

The mechanism is illustrated as follows. Mg+2HF═MgF₂+H₂, wherein: MgF₂ is a compact film, which tightly bonds to the magnesium matrix in a form of chemical bond and is formed on the surface of the magnesium material for avoiding magnesium being further corroded. Therefore, the fluorinated treatment of the magnesium alloy is conventionally an important pretreatment technology of the anti-corrosion treatment for the magnesium alloy. Moreover, titanium or iron reacts with HF that: Ti+6HF→H₂TiF₆+2H₂; 2Fe+12HF→2H₃FeF₆+3H₂, wherein H₂TiF₆ and H₃FeF₆ are both soluble in the hydrofluoric acid and thus pure titanium or pure iron is easily corroded by the hydrofluoric acid.

Fourthly, the open-cell porous material applicable in the field of tissue engineering scaffolds has a good biocompatibility. The mechanical properties of the porous structure match with the biological tissue; and, the open-cell structure is beneficial for the nutrition exchange between the defective tissue and the surrounding tissue, and meanwhile facilitates the ingrowth of the vessel and the surrounding tissue.

### BRIEF DESCRIPTION OF THE DRAWINGS

Through describing the nonrestrictive examples of the present invention in detail with the accompanying drawing, other features, objects and advantages of the present invention will become more apparent.

The drawing is a scanning electron microscope (SEM) photo of a prepared degradable three-dimensional porous magnesium-based biomaterial according to a first example of the present invention.

### DETAILED DESCRIPTION OF THE PREFERRED EMBODIMENT

The present invention is further illustrated in detail with the following examples. The examples will help one skilled in the art further understand the present invention, but not for limiting the present invention in any form. It should be pointed out that modifications and improvements made by one skilled in the art without departing from the spirit of the present invention belong to the protection scope of the present invention.

### EXAMPLE 1

The present invention provides a degradable three-dimensional open-cell porous magnesium alloy which is applicable in a field of tissue engineering, with spherical pores having a pore size of 400-600 μm, wherein: the number of interconnected pores on an inner wall of each pore cavity is 5-7, a pore size of the interconnected pores is 150-250 μm; and a porosity of the alloy is 75%. A structure of the alloy is showed in the drawing, and the spherical pores and the interconnected pores which are uniformly distributed on the pore wall can be seen from the drawing.

According to a first example of the present invention, a preparation method of the degradable three-dimensional open-cell porous magnesium alloy applicable in the tissue engineering comprises steps of:

(1) processing spherical titanium particles having a particle size of 400-600 μm with spark plasma sintering, wherein a sintering temperature is 800° C., a temperature increase rate is 20° C./min, and a sintering pressure is 5 MPa; keeping the sintering temperature and the sintering pressure for 3 minutes, then naturally cooling, and obtaining an open-cell porous titanium-ball preform;

(2) introducing a molten magnesium alloy of Mg-5 wt. % Zn-1 wt. % Mn into gaps of the open-cell porous titanium-ball preform through infiltration casting at a temperature of 720° C. and a pressure of 3 MPa; then decreasing to a room temperature by air cooling, and obtaining a composite compact of the preform and the magnesium alloy; and

(3) on a shaking table, immersing the composite compact into a hydrofluoric acid solution having a weight percentage of 40 wt. % and washing for 6 hours, then washing the composite compact with absolute ethyl alcohol serving as an ultrasonic washing buffer solution for 5 minutes, and repeating washing the composite compact with the hydrofluoric acid solution and the ultrasonic washing buffer solution for 3 times; and obtaining the three-dimensional open-cell porous magnesium alloy with a compressive strength of 2.3 MPa and an elasticity modulus of 0.15 GPa.

### EXAMPLE 2

The present invention provides a degradable open-cell porous magnesium alloy which is applicable in bone tissue engineering scaffolds, with spherical pores having a pore size of 400-600 μm, wherein: the number of interconnected pores on an inner wall of each pore cavity is 4-6, a pore size of the interconnected pores is 250-350 μm; and a porosity of the alloy is 85%.

According to a second example of the present invention, a preparation method of the degradable open-cell porous magnesium alloy applicable in the tissue engineering scaffolds comprises steps of:

(1) processing spherical iron particles having a particle size of 400-600 μm with spark plasma sintering, wherein a sintering temperature is 900° C., a temperature increase rate is 40° C./min, and a sintering pressure is 10 MPa; keeping the sintering temperature and the sintering pressure for 3 minutes, then naturally cooling, and obtaining an open-cell porous iron-ball preform;

(2) introducing a molten magnesium alloy of Mg-3 wt. % Nd-0.2 wt. % Zn-0.5 wt. % Zr-0.5wt. % Ca into gaps of the open-cell porous iron-ball preform through infiltration casting at a temperature of 720° C. and a pressure of 6 MPa; then decreasing to a room temperature by air cooling, and obtaining a composite compact of the preform and the magnesium alloy; and

(3) on a shaking table, immersing the composite compact into a hydrofluoric acid solution having a weight percentage of 40 wt. % and washing for 8 hours, then washing the composite compact with absolute ethyl alcohol serving as an ultrasonic washing buffer solution for 6 minutes, and repeating washing the composite compact with the hydrofluoric acid solution and the ultrasonic washing buffer solution for 7 times; and obtaining the three-dimensional open-cell porous magnesium alloy with a compressive strength of 1.6 MPa and an elasticity modulus of 0.10 GPa.

### EXAMPLE 3

The present invention provides a degradable open-cell porous magnesium which is applicable in tissue engineering scaffolds, with spherical pores having a pore size of 600-800 μm, wherein: the number of interconnected pores on an inner wall of each pore cavity is 4-10, a pore size of the interconnected pores is 350-500 μm; and a porosity of the alloy is 90%.

According to a third example of the present invention, a preparation method of the degradable three-dimensional open-cell porous pure magnesium applicable in the tissue engineering scaffolds comprises steps of:

(1) processing spherical iron particles having a particle size of 600-800 μm with hot isostatic pressing sintering, wherein a sintering temperature is 1000° C., a temperature increase rate is 100° C./min, and a sintering pressure is 50 MPa; keeping the sintering temperature and the sintering pressure for 5 minutes, then naturally cooling, and obtaining an open-cell porous iron-ball preform;

(2) introducing molten pure magnesium into gaps of the open-cell porous iron-ball preform through infiltration casting at a temperature of 720° C. and a pressure of 0.1 MPa; then decreasing to a room temperature by air cooling, and obtaining a composite compact of the preform and the pure magnesium; and

(3) on a shaking table, immersing the composite compact into a hydrofluoric acid solution having a weight percentage of 40 wt. % and washing for 5 hours, then washing the composite compact with absolute ethyl alcohol serving as an ultrasonic washing buffer solution for 5 minutes, and repeating washing the composite compact with the hydrofluoric acid solution and the ultrasonic washing buffer solution for 5 times; and obtaining the three-dimensional open-cell porous magnesium with a compressive strength of 1 MPa and an elasticity modulus of 0.05 GPa.

### EXAMPLE 4

The present invention provides a degradable open-cell porous magnesium alloy which is applicable in tissue engineering scaffolds, with spherical pores having a pore size of 100-400 μm, wherein: the number of interconnected pores on an inner wall of each pore cavity is 4-5, a pore size of the interconnected pores is 50-150 μm; and a porosity of the alloy is 60%.

According to a fourth example of the present invention, a preparation method of the degradable three-dimensional open-cell porous magnesium alloy applicable in the tissue engineering scaffolds comprises steps of:

(1) processing spherical iron particles having a particle size of 100-400 μm with spark plasma sintering, wherein a sintering temperature is 600° C., a temperature increase rate is 10° C./min, and a sintering pressure is 25 MPa; keeping the sintering temperature and the sintering pressure for 1 minute, then naturally cooling, and obtaining an open-cell porous iron-ball preform;

(2) introducing a molten magnesium alloy of Mg-0.4 wt. % As into gaps of the open-cell porous iron-ball preform through infiltration casting at a temperature of 720° C. and a pressure of 10 MPa; then decreasing to a room temperature by air cooling, and obtaining a composite compact of the preform and the magnesium alloy; and

(3) on a shaking table, immersing the composite compact into a hydrofluoric acid solution having a weight percentage of 40 wt. % and washing for 24 hours, then washing the composite compact with absolute ethyl alcohol serving as an ultrasonic washing buffer solution for 15 minutes, and repeating washing the composite compact with the hydrofluoric acid solution and the ultrasonic washing buffer solution for 10 times; and obtaining the three-dimensional open-cell porous magnesium alloy with a compressive strength of 12 MPa and an elasticity modulus of 1.5 GPa.

The examples of the present invention are described as above. It should be understood that the present invention is not limited by above examples, and one skilled in the art can obtain various changes and modifications in the range of the accompanying claims, which will not influence the essential content of the present invention.

1. A preparation method of a three-dimensional interconnected porous magnesium-based material, comprising steps of:
preparing a porous titanium preform or a porous iron preform;
introducing a molten magnesium-based metal into the porous titanium or iron preform through pressure infiltration, and obtaining a porous magnesium-based material precursor; and
washing the porous magnesium-based material precursor, and obtaining the porous magnesium-based material.

2. The preparation method of the three-dimensional interconnected porous magnesium-based material, as recited in claim 1, wherein the porous titanium preform or the porous iron preform is prepared through cold press forming, hot isostatic pressing sintering, microwave sintering or spark plasma sintering.

3. The preparation method of the three-dimensional interconnected porous magnesium-based material, as recited in claim 2, wherein:
preparing the porous titanium preform or the porous iron preform through the spark plasma sintering comprises steps of:
under a pressure of 5-50 MPa, heating titanium particles or iron particles to a temperature of 600-1000° C. with a temperature increase rate of 10-100° C./min; keeping the pressure and the temperature, and then sintering; and, obtaining the porous titanium preform or the porous iron preform.

4. The preparation method of the three-dimensional interconnected porous magnesium-based material, as recited in claim 3, wherein a particle size of the titanium particles or the iron particles is in a range of 10-10000 μm, and the titanium particles or the iron particles have a single particle size or various particle sizes for a mixed use.

5. The preparation method of the three-dimensional interconnected porous magnesium-based material, as recited in claim 1, wherein introducing the molten magnesium-based metal into the porous titanium or iron preform through the pressure infiltration comprises steps of: under a pressure of 0.1-10 MPa, at a temperature of 650-750° C., pouring the molten magnesium-based metal into the porous titanium preform or the porous iron preform, and filling gaps of the porous titanium preform or the porous iron preform with the molten magnesium-based metal.

6. The preparation method of the three-dimensional interconnected porous magnesium-based material, as recited in claim 1, wherein washing the porous magnesium-based material precursor comprises steps of: acid washing through immersing the porous magnesium-based material precursor into a hydrofluoric acid solution, thereafter processing the porous magnesium-based material precursor with ultrasonic washing through an ultrasonic washing buffer solution, and repeating acid washing and ultrasonic washing for at least 3 times.

7. The preparation method of the three-dimensional interconnected porous magnesium-based material, as recited in claim 1, wherein the magnesium-based metal comprises following components by weight percentage of: magnesium: 70-100 wt. %; zinc: 0-30 wt. %; neodymium: 0-5 wt. %; yttrium: 0-10 wt. %; gadolinium: 0-10 wt. %; zirconium: 0-1 wt. %; calcium: 0-2 wt. %; aluminum: 0-9 wt. %; manganese: 0-1 wt. %; and arsenic: 0-2 wt. %.

8. A porous magnesium-based material prepared through the method as recited in claim 1, wherein the porous magnesium-based material has a plurality of cavities therein, and the cavities are intercommunicated with each other through interconnected pores.

9. The porous magnesium-based material, as recited in claim 8, wherein: the interconnected pores of the porous magnesium-based material have a pore size in a range of 2-5000 μm; and the porous magnesium-based material has a porosity of 60-95%, a compressive strength of 1-30 MPa and an elasticity modulus of 0.05-1.5 GPa.

10. (canceled)

11. A method for applying a porous magnesium-based material as recited in claim 1, comprising steps of: applying the porous magnesium-based material in bone tissue engineering scaffolds and other engineering components of a magnesium alloy porous structure requiring characteristics of sound attenuation, sound absorption, noise reduction, shock absorption, thermal insulation, filtration and anti-collision.

12. A method for applying a porous magnesium-based material as recited in claim 8, comprising steps of: applying the porous magnesium-based material in bone tissue engineering scaffolds and other engineering components of a magnesium alloy porous structure requiring characteristics of sound attenuation, sound absorption, noise reduction, shock absorption, thermal insulation, filtration and anti-collision.
